(19) United States
(12) Patent Application Publication
(10) Pub. No.: US 2007/0184116 A1
(43) Pub. Date: Aug. 9, 2007

(54) Pharmaceutical formulation containing unmilled flutamide

(76) Inventors: Hans-Joachim Runge, Hohenwarte (DE); Adalbert Lembcke, Colbitz OT Lindhorst (DE)

Correspondence Address: Ronald R Santucci;Frommer Lawrence & Haug, 745 Fifth Avenue, New York, NY, 10151, US

(21) Appl. No.: 10/593,657

(22) PCT Filed: Mar. 17, 2005
(86) PCT No.: PCT/EP05/02884

§ 371 Date: Apr. 16, 2007

(30) Foreign Application Priority Data
Mar. 22, 2004 (DE) 10 2004 014 272.6

(51) Int. Cl. A61K 31/135; A61K 9/14

(52) U.S. Cl. 424/489; 514/646; 528/482

(57) ABSTRACT

Pharmaceutical formulation comprising crystalline and/or amorphous unmilled flutamide mixed with at least one surface-active substance.

The invention relates to a pharmaceutical formulation containing crystalline and/or amorphous unmilled flutamide and to a process for the preparation thereof.

Flutamide denotes the chemical compound 4′-nitro-3′-trifluoromethylisobutyranilide or 2-methyl-N-[4-nitro-3-(trifluoromethyl)phenyl]propanamide (CAS 13311-84-7). The chemical synthesis of flutamide is described in Baker et al., J. Med. Chem. 10, p. 93 (1967), in U.S. Pat. No. 3,847,988, U.S. Pat. No. 3,995,060, DE 21 30 450 and in DE 22 61 293. Flutamide is a non-steroidal compound that is distinguished especially by anti-androgen properties (cf. Martindale, “The Extra Pharmacopoeia”, 30^{th} edition (1993), p. 482; DE 21 30 450, DE 22 61 293; U.S. Pat. No. 3,847,988). Flutamide is used in the palliative treatment of carcinoma of the prostate. In the case of oral administration, the recommended daily dose is 750 mg. That dose is generally divided over the day, in the form of 3 partial amounts each of 250 mg. In the case of tablets, the recommended amount of active ingredient is usually contained in one tablet; in the case of capsules it is divided over two, each containing 125 mg of active ingredient. Flutamide is a prodrug; its pharmacologically active metabolite is “2-hydroxyflutamide”. Flutamide is virtually insoluble in water and in acid medium, for example in 0.1% hydrochloric acid, the pH value of which corresponds to that of gastric juice. In view of its insolubility in water and the high oral dose required, the bioavailability of flutamide is strongly dependent on the particle size of the active ingredient. That fact can be explained as follows: first, the active ingredient needs to be released from the medicament form and more advantageously dissolved or finely distributed in the digestive juice before it is capable of permeating through the membrane of the mucosae and being taken up by the organism. The preparation of pharmaceutical compositions containing flutamide using especially small particles of active ingredient is described in the literature.

DE 21 30 450 describes the use of flutamide as a veterinary medicine, the active ingredient being used in finely milled form.

U.S. Pat. No. 3,995,060 discloses the preparation of tablets, parenteral suspensions and capsules comprising flutamide. The active ingredient is chemically synthesised, isolated and purified, for example, by recrystallisation (column 2, line 37). As a starting material for the preparation of capsules, the flutamide is mixed with pieces of dry ice and milled to a particle size of from 240 to 5 μm (Table, column 17), then mixed with, inter alia, sodium lauryl sulphate (SLS) in a ratio by weight of flutamide: SLS of from 1.0:1.2 to 1.0:0.06 (Table, column 16), and subsequently subjected to a further milling operation (column 17, lines 17 to 20).

Although according to U.S. Pat. No. 3,995,060, before being combined with any further substance the flutamide, which has been purified by recrystallisation, is in addition subjected to a first milling operation and, after mixing with, inter alia, sodium lauryl sulphate, subjected to a further milling operation, there is criticism in U.S. Pat. No. 6,187,345 (column 1, lines 30 to 43) and U.S. Pat. No. 6,228,401 (column 1, lines 38 to 51) that U.S. Pat. No. 3,995,060 has not considered the active ingredient comminution sufficiently critically with a view to adequate bioavailability. U.S. Pat. No. 6,187,345 and U.S. Pat. No. 6,228,401 describe the preparation of flutamide particles having a specific surface area of from 0.40 to 2.50 m²/cm³ (column 2, line 67 and column 3, line 7, respectively) and a particle size of from 10 to 130 μm (column 3, line 4 and column 3 lines 11 to 12, respectively). For that purpose, flutamide is milled in the presence of, for example, lactose as diluent (Examples 1 to 4). The diluent prevents agglomeration of the flutamide during the milling process. The micronised flutamide so obtained, having a particular particle size and specific surface area, should exhibit adequate bioavailability. For the preparation of capsules, the resulting mixture of flutamide and lactose is granulated in moist state with, inter alia, sodium lauryl sulphate (SLS) (Examples 6, 7 and 9), the ratio by weight of flutamide: SLS being from 1.0:0.096 to 1.0:0.06.

In “Dissolution rate limited bioavailability of flutamide, and in vitro—in vivo correlation”, J. Post et al., Eur. J. Pharm. Biopharm., 49 (2000), 35-39, it is demonstrated that the use of unmilled flutamide in tablets yields an active-ingredient release of 45% after 45 min. whereas, when finely milled flutamide is used, a rate of release of 90% is achieved in the same time. It is furthermore demonstrated that the release of the active ingredient is, in vitro, quantitatively correlated with its bioavailability. As a measure thereof, the AUC value of the metabolite that is actually active, 2-hyroxyflutamide, is used. This is a generally recognised procedure so that, in determining the in vitro values, it is possible to draw reliable conclusions in respect of the bioavailability of the flutamide in its preparations. An examination of 25 batches of 250 mg flutamide tablets from different manufacturers from the period from 1987 to 1994 showed that those products vary in their respective rates of release (after 45 min.) within a range of from 47 to 94%. In addition, the rates of release of different batches from the same manufacturer vary within from 47 to 86%. In view of the wide variation in values for the rates of release, the preparation processes hitherto cannot be described as valid. Adequate clinical reliability is not possible. The reason for that is that the substance properties of flutamide are difficult to control.

In Europ. J. Pharm. Biopharm., 49 (2000) 35-39, Posti et al. describe conventional tablets containing milled or unmilled flutamide for the direct release of active ingredient and with a content of maize starch, microcrystalline cellulose, anhydrous colloidal silica and magnesium stearate.

Finally, capsules that contain unmilled flutamide are described in U.S. Pat. No. 6,228,401 (Test Procedure I). The concentration of active ingredient achieved in plasma therewith is compared with a concentration that is achievable with Eulexin® as a commercial product.

A disadvantage of a milling process for flutamide is the cost-intensive cooling required for the milling procedure. The process heat generated during the milling of flutamide needs to be discharged, since otherwise melting or sintering phenomena may arise because of the low melting point of flutamide (111° C.). Although the preparation of micronised flutamide, for example, in air-jet mills, is possible with large expenditure on cooling, the flutamide milled in that manner tends to form agglomerates. Accordingly, also prolonged storage of micronised flutamide is possible only with limitations.

The problem of the invention is the provision of pharmaceutical formulations containing crystalline and/or amorphous unmilled flutamide. The formulations are to have a reproducible and high rate of release of the active ingredient. The preparation of those formulations is to be economic.

According to one embodiment, the problem underlying the invention is solved by a pharmaceutical formulation comprising crystalline and/or amorphous unmilled flutamide mixed with at least one surface-active substance.

Thus, the invention relates to a pharmaceutical formulation comprising crystalline and/or amorphous unmilled flutamide mixed with at least one surface-active substance, wherein the formulation is in the form of a tablet with a content of at least one flow regulator.

Thus, the invention relates also to a pharmaceutical formulation comprising crystalline and/or amorphous unmilled flutamide mixed with at least one surface-active substance, wherein the formulation is in the form of a filling for capsules.

Thus, the invention relates also to a pharmaceutical formulation comprising crystalline and/or amorphous unmilled flutamide mixed with at least one surface-active substance, wherein the formulation is in the form of a dragee, effervescent tablet, suppository or granulate.

The formulation according to the invention may be provided with flutamide of a particle size such as is formed in the synthesis process with subsequent purification step(s).

Further, the formulation according to the invention may be provided with flutamide of a particle size such as is formed in the synthesis process with recrystallisation as a subsequent purification step.

Further, the formulation according to the invention may be provided with flutamide of a mean particle size greater than the mean particle size of flutamide that, with an initial particle size of from 5 to 240 μm, has been subjected to a milling operation.

Further, the formulation according to the invention may be provided with flutamide in which the size of 50% of the flutamide particles (X50 value) is greater than 20 μm.

Further, the formulation according to the invention may be provided with flutamide in which the size of 50% of the flutamide particles (X50 value) is greater than 26 μm.

Further, the formulation according to the invention may be provided with flutamide in which the size of 90% of the flutamide particles (X90 value) is greater than 60 μm.

Further, the formulation according to the invention may be provided with flutamide in which the size of 90% of the flutamide particles (X90 value) is greater than 130 μm.

Further, the formulation according to the invention may be provided with flutamide having a specific surface area of less than 2.50 m²/cm³.

Further, the formulation according to the invention may be provided with flutamide having a specific surface area of less than 1.50 m²/cm³.

Further, the formulation according to the invention may be provided with flutamide having a specific surface area of less than 0.35 m²/cm³.

Further, the formulation according to the invention may be provided with flutamide and/or one of its pharmaceutically acceptable solvates.

Further, the formulation according to the invention may be provided with at least one surface-active substance selected from the group formed by
anionic compounds,
cationic compounds and
non-ionic surfactants.

Further, the formulation according to the invention may be provided with sodium dodecylsulphate as surface-active substance.

Further, the formulation according to the invention may be provided with a ratio by weight of flutamide: surface-active substance(s) of from 5:1 to 30:1.

Further, the formulation according to the invention may be provided with a ratio by weight of flutamide: surface-active substance(s) of from 5:1 to 20:1.

Further, the formulation according to the invention may be provided with a ratio by weight of flutamide: surface-active substance(s) of from 10:1 to 15:1.

Further, the formulation according to the invention may be provided in the form of an unshaped mixture or in the form of an article that has been subjected to shaping, for example in the form of a tablet, a capsule filling, a dragee, an effervescent tablet, a suppository or a granulate.

Further, the formulation according to the invention may be provided with a content of from 50 to 2000 mg of flutamide.

Further, the formulation according to the invention may be provided with a content of from 50 to 500 mg of flutamide.

Further, the formulation according to the invention may be provided with a content of from 100 to 200 mg of flutamide.

Further, the formulation according to the invention may be provided with a content of at least one excipient from the group formed by inorganic fillers, organic fillers, binders, glidants, lubricants, flow regulators and/or disintegrants.

Further, the formulation according to the invention may be provided with a content of at least one further pharmaceutical active ingredient besides flutamide.

According to a further embodiment, the problem underlying the invention is solved by a process for the preparation of a pharmaceutical formulation comprising crystalline and/or amorphous unmilled flutamide mixed with at least one surface-active substance, in which the flutamide is subjected to an intensive mixing process with the at least one surface-active substance, preferably mixed in a forced-action mixer, and the mixture obtained is further processed to form a formulation according to the invention.

In the process according to the invention, mixing may be carried out at a temperature of from 0 to 40° C.

Further, in the process according to the invention mixing may be carried out at room temperature.

Further, in the process according to the invention one or more excipients may be admixed using a forced-action mixer or using a free-fall mixer.

Further, in the process according to the invention one or more further pharmaceutical active ingredients, which are not flutamide, may be admixed using a forced-action mixer or a free-fall mixer.

Further, the mixture obtained in the process according to the invention may be further processed to form tablets, capsules, dragees, effervescent tablets, suppositories or a granulate.

Further, the mixture obtained in the process according to the invention may be subject to direct tableting.

Finally, in the process according to the invention a granulate may be further processed to form tablets.

The problem underlying the invention is in addition solved by a pharmaceutical formulation comprising crystalline and/or amorphous unmilled flutamide mixed with at least one surface-active substance, which formulation can be prepared in accordance with a process according to the invention.

Surprisingly, it has thus been found that it is possible for unmilled flutamide to be processed to form pharmaceutical formulations with high and reproducible rates of release when the formulation is based on a powder mixture comprising crystalline and/or amorphous flutamide and at least one surface-active substance. To produce the powder mixture according to the invention, unmilled flutamide, at least one surface-active substance and, where appropriate, further excipients, are intensively mixed in a forced-action mixer. That powder mixture may be further processed, for example to form tablets, capsules, dragees, effervescent tablets or suppositories. The pharmaceutical formulations so obtained exhibit not only a reproducible, but also a higher, rate of release of flutamide than formulations comprising micronised active ingredient. The preparation with the aid of a forced-action mixer is more economical and time-saving compared with an additional expensive milling process with loss of material.

For the pharmaceutical formulation according to the invention, flutamide may be used in the form of the free acid amide and/or one of its solvates.

The daily dose of flutamide is from 2 to 30 mg per kg body weight of the patient, preferably from 7 to 14 mg/kg body weight. A daily dose may be divided into several administrations per day. A pharmaceutical formulation may contain from 100 to 2000 mg of flutamide.

The expression “unmilled flutamide” is understood to mean crystalline or amorphous flutamide, which is usually obtained in a purification procedure (e.g. recrystallisation) subsequent to the synthesis.

Suitable surface-active substances are:
anionic compounds, such as sodium dodecylsulphate or sodium docusate [stearates are understood in the context of the invention as glidants, lubricants or flow regulators; cf. also Hunnius, Pharmazeutisches Wörterbuch, 8^{th} edition, De Gruyter, for the terms Gleitmittel, Schmiermittel and Flieβmittel or Flieβreguliermittel (glidants, lubricants and flow agents or flow regulators)],
cationic compounds, such as triethanolamine oleate,
non-ionic surfactants, for example cetyl alcohol, polysorbate, glyceryl monostearate, glyceryl monooleate, polyvinyl alcohol, Tweens® such as sorbitan monoisostearate, sorbitan monolaurate, sorbitan monopalmitate, sorbitan monostearate, sorbitan monooleate, sorbitan sesquioleate or sorbitan trioleate.

It is also possible to use mixtures containing a plurality of surfactants.

Preference is given to the use of sodium dodecylsulphate.

The ratio by weight of flutamide to the surface-active substance(s) is from 5:1 to 30:1, especially from 10:1 to 15:1.

The following excipients may be used for the preparation of the powder mixture:
fillers, such as cellulose (e.g. microcrystalline cellulose), cellulose derivatives, sugars (e.g. lactose, glucose, saccharose), sugar alcohols (e.g. mannitol, sorbitol), starch (e.g. potato starch, wheat starch, maize starch and/or rice starch),
glidants, such as silica, calcium stearate, aluminium stearate and/or magnesium stearate,
lubricants, such as aluminium stearate, magnesium stearate or calcium stearate, stearic acid, sodium stearylfumarate, polyethylene glycol, stearyl alcohol or cetyl alcohol, palmitic acid, hydrogenated vegetable oils and/or talc,
disintegrants, such as starch (e.g. potato starch or maize starch), polyvinylpyrrolidones (collidones, crosslinked polyvinylpyrrolidone), unmodified or modified cellulose (e.g. microcrystalline cellulose, sodium carboxymethylcellulose, crosslinked carboxymethyl-cellulose)ultraamylopectin, formaldehyde gelatin and/or alginic acid or its alginates.

When the powder mixture according to the invention is used to fill capsules, then preferably the following excipients are used to prepare the powder mixture:
lactose, microcrystalline cellulose and maize starch as fillers,
magnesium stearate and silica as glidants.

When the powder mixture according to the invention is further processed to form tablets, then preferably the following excipients are used to prepare the powder mixture:
lactose and microcrystalline cellulose as fillers,
maize starch and microcrystalline cellulose as disintegrants,
silica as glidant
magnesium stearate as lubricant and flow regulator.

For the preparation of the powder mixture according to the invention, unmilled flutamide is subjected to an intensive mixing process in a forced-action mixer with at least one surface-active substance. Further excipients may be admixed in an additional free-fall mixer or in the same forced-action mixer, in succession or simultaneously.

Unmilled flutamide may also be intensively mixed in a forced-action mixer together with at least one surface-active substance and one or more excipients.

Conventional forced-action mixers with a stainless steel interior may be used for the process according to the invention. Forced-action mixers are generally mixers having a round or flat base and blades or paddles rotating close to the base. They may have so-called “choppers”, that is, rapidly rotating knives, which project into the mixing vessel. The speed of rotation of the blades may be from 100 to 800 revs/min, and that of the “choppers” from 750 to 3000 revs/min. Preferably, the speed of rotation used for the blades is from 150 to 200 revs/min and that for the “choppers” is 1500 revs/min. The forced-action mixer used may be, for example, a Collette Vactron® mixer, Lödige® mixer or Diosna® mixer or a Bohle-Vagumat®.

The forced-action mixing process may be carried out at various temperatures but, for economic reasons, it is preferably carried out at room temperature. The process time may be from 1 to 180 min., especially from 3 to 60 min.

The powder mixture according to the invention comprising unmilled flutamide may be used to prepare pharmaceutical forms of administration, for example capsules, tablets, effervescent tablets, dragees, suspensions or suppositories. Those forms of administration may also comprise excipients, such as carriers, fillers, binders, humectants, glidants, preservatives, stabilisers, flavourings and/or colouring pigments.

Further Processing to Form Capsules:

The powder mixture according to the invention comprising unmilled flutamide may be filled into capsules.

Further Processing to Form Tablets:

The powder mixture according to the invention comprising unmilled flutamide may be subject to direct tableting. It may, however, also be processed to form granulates which, mixed with further excipients, may be compressed to form tablets.

Suitable excipients in the preparation of granulates and tablets are, for example:
organic fillers, such as cellulose and/or cellulose derivatives (e.g. microcrystalline cellulose), sugars (e.g. lactose, glucose, saccharose), sugar alcohols (e.g. mannitol, sorbitol), starch (e.g. potato starch, wheat starch, maize starch and/or rice starch),
inorganic fillers, such as calcium phosphate or calcium sulphate, sodium calcium phosphate, sodium chloride or kaolin,
binders, such as gelatin derivatives, starch derivatives or cellulose derivatives (e.g. hydroxypropyl cellulose, hydroxypropyl methylcellulose, methylcellulose, sodium carboxymethylcellulose), polyvinylpyrrolidones, sugars (e.g. sucrose, glucose, dextrose), natural rubber (e.g. sodium alginate, arabic gum, tragacanth, pectin),
lubricants, such as magnesium stearate, calcium stearate, stearic acid, sodium stearylfumarate, polyethylene glycol, palmitic acid, hydrogenated vegetable oils and/or talc,
flow regulators, such as talc, stearic acid and its alkaline earth metal salts, silica, polyethylene glycols and long-chained alcohols,
disintegrants, such as starch and starch derivatives (maize starch, sodium carboxymethyl starch), crosslinked polyvinylpyrrolidone, unmodified or modified cellulose, (e.g. sodium carboxymethylcellulose, crosslinked carboxymethylcellulose), alginic acid and its alginates, calcium carbonate and/or sodium hydrogen carbonate, surface-active substances (e.g. syndets).

The tablets may be coated with a film-forming material, for example to achieve resistance to gastric juice, obtain a smooth surface or enhance the stability of the tablet during packaging and transport. The tablet coating may comprise, for example, additives such as “anti-tacking” materials or colourants.

Release Profile:

In vitro release studies for capsules or tablets comprising flutamide were carried out using a Vankel apparatus with a blade agitator. A 2% sodium lauryl sulphate solution at a pH value of from 5.5 to 7.5 was used as the release medium.

The invention is explained in greater detail by the Examples hereinafter, but without the scope of the invention being limited thereby.

### EXAMPLE 1

The following substances are used to prepare flutamide capsules:

| Constituents | Percentage (%) | Weight (mg/capsule) |
|---|---|---|
| flutamide, crystalline, unmilled | 33.2 | 80.0 |
| lactose | 29.4 | 70.9 |
| sodium dodecylsulphate | 2.0 | 4.8 |
| microcrystalline cellulose | 13.26 | 32.0 |
| maize starch | 21.6 | 52.0 |
| silica | 0.04 | 0.1 |
| magnesium stearate | 0.5 | 1.2 |
| total | 100 | 241 |

Preparation:

Lactose, sodium dodecylsulphate, microcrystalline cellulose and crystalline flutamide are intensively mixed for 3 min. in a forced-action mixer. Maize starch, colloidal silica and magnesium stearate are then added and the mixture is homogenised for 5 min. in a free-fall mixer. That mixture is filled into hard gelatin capsules in an amount of 241 mg per capsule.

### EXAMPLE 2 (COMPARISON)

The following substances are used to prepare flutamide capsules

| Constituents | Percentage (%) | Weight (mg/capsule) |
|---|---|---|
| flutamide, micronised | 33.2 | 80.0 |
| lactose | 29.4 | 70.9 |
| sodium dodecylsulphate | 2.0 | 4.8 |
| microcrystalline cellulose | 13.26 | 32.0 |
| maize starch | 21.6 | 52.0 |
| silica | 0.04 | 0.1 |
| magnesium stearate | 0.5 | 1.2 |
| total | 100 | 241 |

Preparation:

Lactose, sodium dodecylsulphate, microcrystalline cellulose, maize starch, colloidal silica and magnesium stearate are mixed with micronised flutamide for 15 min. in a free-fall mixer. That mixture is filled into hard gelatin capsules in an amount of 241 mg per capsule.

### EXAMPLE 3

The following substances are used to prepare flutamide capsules:

| | | Weight |
|---|---|---|
| Constituents | Percentage (%) | (kg/100,000 capsules) |
| flutamide, crystalline, unmilled | 33.0 | 12.446 |
| lactose | 29.3 | 11.047 |
| sodium dodecylsulphate | 2.0 | 0.747 |
| microcrystalline cellulose | 13.2 | 4.981 |
| maize starch | 21.9 | 8.272 |
| silica | 0.1 | 0.020 |
| magnesium stearate | 0.5 | 0.188 |
| total | 100 | 37.701 |

Preparation:

Lactose, sodium dodecylsulphate, microcrystalline cellulose and crystalline flutamide are intensively mixed for 40 min. in a forced-action mixer. Maize starch, colloidal silica and magnesium stearate are then added and the mixture is homogenised for 3 min. at reduced speed. That mixture is filled into hard gelatin capsules in an amount of 377 mg per capsule.

### EXAMPLE 4

The following substances are used to prepare flutamide tablets:

| Constituents | Percentage (%) | Weight (kg) |
|---|---|---|
| flutamide, crystalline, unmilled | 33.2 | 1.658 |
| lactose | 29.4 | 1.471 |
| sodium dodecylsulphate | 2.0 | 0.100 |
| microcrystalline cellulose | 13.2 | 0.663 |
| maize starch | 21.6 | 1.08 |
| silica | 0.1 | 0.003 |
| magnesium stearate | 0.5 | 0.025 |
| total | 100 | 5.000 |

Preparation:

Crystalline flutamide, lactose, sodium dodecylsulphate and microcrystalline cellulose are force-mixed for 10 min. in a Diosna mixer. Maize starch is then added, and forced-action mixing is carried out again for 5 min. After the addition of silica and magnesium stearate, final mixing is carried out for 3 min.

Tablets each weighing 750 mg are pressed directly from that mixture on a rotary tableting machine (hardness 98 N, abraded material <1%).

### EXAMPLE 5 COMPARISON

The following substances are used to prepare flutamide tablets:

| Constituents | Percentage (%) | Weight (kg) |
|---|---|---|
| flutamide, crystalline, unmilled | 33.0 | 21.250 |
| lactose | 54.8 | 18.850 |
| sodium dodecylsulphate | 2.0 | 1.275 |
| microcrystalline cellulose | 13.2 | 8.500 |
| maize starch | 21.5 | 13.820 |
| silica | 0.5 | 0.340 |
| magnesium stearate | 0.5 | 0.340 |
| total | 100 | 64.375 |

Preparation:

Lactose, sodium dodecylsulphate, microcrystalline cellulose, maize starch and crystalline flutamide are granulated with 21.080 litres of water, dried, sieved, and then mixed with colloidal silica and magnesium stearate and compressed to form tablets.

### EXAMPLE 6 (COMPARISON)

The following substances are used to prepare flutamide tablets:

| Constituents | Percentage (%) | Weight (kg) |
|---|---|---|
| flutamide, micronised | 33.0 | 21.250 |
| lactose | 54.8 | 18.850 |
| sodium dodecylsulphate | 2.0 | 1.275 |
| microcrystalline cellulose | 13.2 | 8.500 |
| maize starch | 21.5 | 13.820 |
| silica | 0.5 | 0.340 |
| magnesium stearate | 0.5 | 0.340 |
| total | 100 | 64.375 |

The tablets are prepared analogously to Example 5.

### RATES OF RELEASE FOR EXAMPLES 1-6

The rate of release of flutamide from the pharmaceutical formulations comprising
unmilled flutamide intensively mixed with sodium dodecylsulphate (Examples 1, 3, 4),
micronised flutamide, not intensively mixed with sodium dodecylsulphate (Example 2), quantitative ratios as in Example 1,
unmilled flutamide, not intensively mixed with sodium dodecylsulphate (Example 5),
micronised flutamide, not intensively mixed with sodium dodecylsulphate (Example 6), quantitative ratios as in Example 5,
   was determined.

Apparatus for determining the release of active ingredient:

Vankel apparatus with blade agitator

Cell: 1000 ml

Medium: 2% sodium dodecylsulphate solution

Temperature: 37° C.

Agitating speed: 75 revs/min.

| | | Time/ | Released active |
|---|---|---|---|
| | Processing | min. | ingredient in % |
| Example 1 | unmilled flutamide, | 30 | 100 |
| | intensively mixed | | |
| Example 2 | micronised flutamide, | 30 | 71 |
| | not intensively mixed, | | |
| | same composition as | | |
| | in Example 1 | | |
| Example 3 | unmilled flutamide, | 60 | 93 |
| | intensively mixed | | |
| Example 4 | unmilled flutamide, | 30 | 92 |
| | intensively mixed | | |
| Example 5 | unmilled flutamide, | 30 | 51 |
| | not intensively mixed | | |
| Example 6 | micronised flutamide, | 30 | 87 |
| | not intensively mixed, | | |
| | quantitative ratios | | |
| | as in Example 5 | | |

As may be seen from the Table, the highest rate of release is obtained in the case of a formulation comprising unmilled flutamide that has been intensively mixed with a surface-active substance.

Examples 1 and 2 show that the use of micronised flutamide without the intensive mixing process according to the invention results in a lower rate of release than the use of unmilled flutamide that has been intensively mixed with a surface-active substance.

Examples 5 and 6 confirm the observation known from the literature that a higher rate of release can be achieved using micronised active ingredient compared with unmilled active ingredient. The tablets were prepared according to known granulating methods without forced-action mixing of the constituents.

1. Pharmaceutical formulation comprising crystalline and/or amorphous unmilled flutamide mixed with at least one surface-active substance.

2. Pharmaceutical formulation according to claim 1 comprising crystalline and/or amorphous unmilled flutamide mixed with at least one surface-active substance, wherein the formulation is in the form of a tablet with a content of at least one flow regulator.

3. Pharmaceutical formulation according to claim 1 comprising crystalline and/or amorphous unmilled flutamide mixed with at least one surface-active substance, wherein the formulation is in the form of a filling for capsules.

4. Pharmaceutical formulation according to claim 1 comprising crystalline and/or amorphous unmilled flutamide mixed with at least one surface-active substance, wherein the formulation is in the form of a dragee, effervescent tablet, suppository or granulate.

5. Formulation according to claim 1 comprising flutamide of a particle size such as is formed in the synthesis process with subsequent purification step(s).

6. Formulation according to claim 5 comprising flutamide of a particle size such as is formed in the synthesis process with recrystallisation as a subsequent purification step.

7. Formulation according to claim 1 comprising flutamide of a mean particle size greater than the mean particle size of flutamide that, with an initial particle size of from 5 to 240 μm, has been subjected to a milling operation.

8. Formulation according to claim 1 comprising flutamide in which the size of 50% of the flutamide particles (X50 value) is greater than 20 μm.

9. Formulation according to claim 8 comprising flutamide in which the size of 50% of the flutamide particles (X50 value) is greater than 26 μm.

10. Formulation according to claim 1 comprising flutamide in which the size of 90% of the flutamide particles (X90 value) is greater than 60 μm.

11. Formulation according to claim 10 comprising flutamide in which the size of 90% of the flutamide particles (X90 value) is greater than 130 μm.

12. Formulation according to claim 1 comprising flutamide of a specific surface area of less than 2.50 m²/cm³.

13. Formulation according to claim 12 comprising flutamide of a specific surface area of less than 1.50 m²/cm³.

14. Formulation according to claim 13 comprising flutamide of a specific surface area of less than 0.35 m²/cm³.

15. Formulation according to claim 1 comprising flutamide in the form of the free acid amide and/or of a pharmaceutically acceptable solvate.

16. Formulation according to claim 1 comprising at least one surface-active substance selected from the group formed by
anionic compounds,
cationic compounds and
non-ionic surfactants.

17. Formulation according to claim 16 comprising sodium dodecylsulphate as surface-active substance.

18. Formulation according to claim 1 with a ratio by weight of flutamide: surface-active substance(s) of from 5:1 to 30:1.

19. Formulation according to claim 18 with a ratio by weight of flutamide: surface-active substance(s) of from 5:1 to 20:1.

20. Formulation according to claim 19 with a ratio by weight of flutamide: surface-active substance(s) of from 10:1 to 15:1.

21. Formulation according to claim 1 in the form of an unshaped mixture or in the form of an article that has been subjected to shaping.

22. Formulation according to claim 21 with a content of from 50 to 2000 mg of flutamide.

23. Formulation according to claim 22 with a content of from 50 to 500 mg of flutamide.

24. Formulation according to claim 23 with a content of from 100 to 200 mg of flutamide.

25. Formulation according to claim 1 with a content of at least one excipient from the group formed by inorganic fillers, organic fillers, binders, glidants, lubricants, flow regulators and/or disintegrants.

26. Formulation according to claim 1 with a content of at least one further pharmaceutical active ingredient besides flutamide.

27. Process for the preparation of a pharmaceutical formulation according to claim 1 comprising crystalline and/or amorphous unmilled flutamide mixed with at least one surface-active substance, in which the flutamide is subjected to an intensive mixing process with the at least one surface-active substance.

28. Process for the preparation of a pharmaceutical formulation according to claim 2 comprising crystalline and/or amorphous unmilled flutamide mixed with at least one surface-active substance, in which the flutamide is subjected to an intensive mixing process with the at least one surface-active substance, and the mixture obtained is further processed to form a formulation according claim 2.

29. Process according to claim 27 in which mixing is carried out at a temperature of from 0 to 40° C.

30. Process according to claim 29 in which mixing is carried out at room temperature.

31. Process according to claim 27 in which one or more excipients are admixed using a forced-action mixer or using a free-fall mixer.

32. Process according to claim 27 in which one or more further pharmaceutical active ingredients, which are not flutamide, are admixed using a forced-action mixer or a free-fall mixer.

33. Process according to claim 27 in which the mixture obtained is further processed to form tablets, capsules, dragees, effervescent tablets, suppositories or a granulate.

34. Process according to claim 28 in which the mixture obtained is subject to direct tableting.

35. Process according to claim 28 in which the granulate is further processed to form tablets.

36. Pharmaceutical formulation comprising crystalline and/or amorphous unmilled flutamide mixed with at least one surface-active substance, producible in accordance with a process according to claim 27.
